(19) United States
(12) Patent Application Publication
(10) Pub. No.: US 2007/0197892 A1
(43) Pub. Date: Aug. 23, 2007

(54) NON-PLANAR MULTI-CHANNEL ELECTRODE ARRAY

(75) Inventors: Jessie Y. Shen, London (CA); Souhile Assaf, London (CA); Mandar Jog, London (CA)

Correspondence Address: BORDEN LADNER GERVAIS LLP, WORLD EXCHANGE PLAZA, 100 QUEEN STREET SUITE 1100, OTTAWA, ON, K1P 1J9, CA

(73) Assignee: MEDTRODE INC., London (CA)

(21) Appl. No.: 11/678,100

(22) Filed: Feb. 23, 2007

Related U.S. Application Data
(60) Provisional application No. 60/775,791, filed on Feb. 23, 2006.

(51) Int. Cl. A61B 5/04

(52) U.S. Cl. 600/378; 600/393

(57) ABSTRACT

A multi-channel neuromodulation electrode assembly has multiple electrodes arranged in a non-linear, non-planar fashion by stacking electrode elements to form an electrode stack. Multiple electrode stacks may be combined to produce electrode shanks, and multiple electrode shanks may be combined to produce electrode arrays. This provides an easily customizable non-linear and non-planar channel arrangement. Complex electrode configurations can be assembled from multiple electrode elements. Depending on the requirements, the number of channels, the channel spacing, the complexity of assembly differs. In one embodiment, referred to as a “Christmas tree” configuration, the electrode has two stacks facing opposite directions. In another embodiment, referred to as an “Empire State Building” configuration, the electrode has four stacks facing different directions. In yet another embodiment, the electrode has eight stacks facing different directions. In yet another embodiment, the electrode has eight shanks by eight shanks, where each shank has 14 channels. In yet another embodiment, the electrode has three shanks by three shanks, where each shank has 9 channels.

### REFERENCE TO RELATED APPLICATION

This is a formal application based on and claiming priority from U.S. provisional application No. 60/775,791, filed Feb. 23, 2006.

### BACKGROUND OF THE INVENTION

This invention relates to electrodes used in neuromodulation and brain-computer interface.

Neuromodulation is defined as “the therapeutic alteration of activity in the central, peripheral or autonomic nervous systems, electrically or pharmacologically, by means of implanted devices” by the International Neuromodulation Society. For example, deep brain stimulation (DBS) is an electrical therapy currently used to control tremor in Parkinson patients. Other electrical therapies include spinal cord stimulation (SCS) and vagus nerve stimulation (VNS).

Neuromodulation employs a variety of electrodes depending on the neurophysiological characteristics of the target neuronal nuclei, as well as their particular function. Electrodes commonly serve 3 purposes:
1. stimulation, which means the electrodes deliver the electrical therapy such as DBS;
2. recording, which monitors the neuronal activity, and in more recent development, provides feedback for closed-loop adaptive therapy; and
3. targeting, which identifies the precise location of the target nucleus during the implantation procedure.

Typically, electrodes have only a single purpose such that they are designed for functional optimization. For instance in DBS, a targeting electrode is inserted to identify the target nucleus, a macro-stimulation electrode is inserted to validate the site, and finally an implantable stimulation electrode is implanted. Multiple stimulation channels enable bi-polar stimulation. They also provide the flexibility of selecting a channel close to the nucleus when targeting was not accurate. For recording electrodes, multiple channels enable the simultaneous recording from a volume of tissue.

The functional distinction between electrodes is due to the difference in electrical requirements. For instance, the stimulation electrode needs to pass significant current without damaging the surrounding tissue, which means significantly large contact sizes. However, the recording electrode used to record local potentials needs a much smaller contact size as to isolate one neuron's activity. Thus, electrodes with dedicated function are normally used.

Using multiple electrodes means that the multiple insertions increases the duration of the surgical procedure, raises the likelihood of trauma, introduces the possibility of losing the target site, etc. While electrically independent tracks on a physical electrode body can be customized for particular functions, traditional manufacturing methods would produce an electrode significantly larger in diameter, potentially causing even more trauma in the implantation process.

More recent progress in micro-manufacturing enables multi-functional electrodes with functionally dedicated tracks. Multi-channel electrodes which combine recording and stimulating functions are documented in U.S. Pat. No. 7,010,356 and in United States patent application nos. 2005/246,004 and 2006/026,503. An electrode with both recording and stimulating capabilities would be advantageous because it requires fewer insertions and shorter surgery time. Furthermore, the target site identified by the recording channel will be stimulated directly by a nearby stimulation channel. Also, an electrode with both recording and stimulation capabilities enables adaptive therapy where the feedback is collected through the recording channels.

One of the major problems with stimulating electrodes is the shape of stimulation fields. If the stimulation field spills into neighboring cell clusters, serious undesirable side effects will occur. If the stimulation field is too small, the target cells are not adequately stimulated so no therapeutic effect is gained. The flexibility of the stimulation field is somewhat improved by more channels and directional channels as described in United States patent application no. 2005/015,130, but the range of freedom is limited to the spatial arrangement of the channels.

For any application where chronic recording is required, one problem is tissue adhesion (glial scarring). The buildup of tissue acts as a barrier to the actual active cells that should be recorded. This tissue therefore increases the impedance of the recording channel, eventually making the electrode useless.

Several methods to counter this problem are in research or in testing:
1. One method is called rejuvenation, which periodically sends a very small amplitude signal to the recording site such that the tissue does not adhere to the site. This method has met with limited success.
2. Another method is to mechanically shake the electrode free. However, large movements would lose the recording site and small movement would merely drag the adhered tissue along as opposed to completely detaching from it. The movement may also damage the neurons to be recorded.
3. Another option is to design the recording site such that tissue does not directly adhere to it.

A concrete way to improve the longevity of a recording electrode is offering more channels. The lifetime of each site is different and follows some probability models. A subset of the sites will continue to function long after other ones have failed. Therefore, offering more sites per electrode array will prolong the utility lifetime of the electrode array. It is important, therefore, to have a spatial channel arrangement capable of representing the activity of the entire volume as long as possible.

Another problem for recording electrodes is the movement of the sites relative to the brain. It is difficult to determine the exact movement and compensate for the movement mechanically. Therefore, one solution is having multiple channels such that a neighboring channel can be selected if it provides a clearer recording. This selection process can be accomplished in signal processing by looking for the best recorded field potentials. Using the proper spatial arrangement of the channels, the movement can be deduced by tracking certain neuronal activity using spatial mapping algorithms. Then the movement can be correspondingly compensated.

In addition to neuromodulation, multi-channel electrode arrays are also used for brain computer interface (BCI), where the electrodes record at many sites to map the subject's neuronal activity and to correlate to the subject's intention. The signals are recorded in application specific regions of the cortex. For instance, to move a cursor, the signals are collected from the motor cortex. BCI needs the channels to be densely covering a 3D volume of neuronal tissue, or at the very least a 2D volume of neuronal tissue.

Currently, multi-channel electrodes use linear or planar channel arrangements. In a linear arrangement, a straight line can be drawn through the centers of all the contact pads. A planar arrangement means that a plane passes through all the contact pads.

Medtronic Inc. builds a deep brain stimulation electrode that has 4 cylindrical channels linearly arranged along the central axis of the cylinders. The stimulation fields achieved with this electrode are limited to a sphere or an ovoid, whereas the target neuronal nuclei often have irregular shapes. The shape limitations of the stimulation field either causes inadequate coverage which reduces the effectiveness of the therapy, or causes spillage into neighboring nuclei which causes undesirable side-effects.

The multi-channel probes described in U.S. Pat. Nos. 6,330,466 and 6,829,498 have planar surface on which all the channels are arranged. For certain applications these planar channels are further positioned into a linear formation. When recording from a linear or planar channel arrangement, the source of activity can only be deduced as a projection on the line or the plane, respectively. A 3D spatial location can only be identified by a set of recording channels that can define the 3D volume.

The electrode described in U.S. Pat. No. 5,214,088 is a comb-shaped electrode array used for brain-computer interface. The electrode is implanted in the cortex to record the subject's intentions. This electrode is an array of insulated pins whose exposed tips form a planar arrangement of channels, which means it cannot record from a volume of neurons.

Both linear and planar channel arrangements lack the ability to properly represent a volume of neuronal tissue. Therefore, they cannot stimulate in such a way as to properly control the volume of tissue. Furthermore, the spatial location of certain neuronal activity cannot be deduced from the data recorded from such electrodes.

U.S. Pat. No. 7,010,356 describes a non-planar arrangement of channels. The primary embodiment places the channels on a substantially cylindrical substrate, and has a non-planar arrangement of channels because the channels have differences in direction and in height. However, these designs lack the flexibility for other electrode shapes. Furthermore, customization in terms of spacing and geometry will take longer because the entire design needs to be modified.

### SUMMARY OF THE INVENTION

The invention provides a multi-channel neuromodulation electrode assembly, comprising multiple electrodes arranged in a non-linear, non-planar fashion by stacking electrode elements to form an electrode stack.

The invention preferably also provides electrode shanks, comprising multiple electrode stacks produced according to the invention.

The invention preferably further provides electrode arrays, comprising multiple electrode shanks produced according to the invention.

An advantage of the invention is that it provides a multi-channel neuromodulation electrode array with an easily customizable non-linear and non-planar channel arrangement. For stimulation and recording, this enables, respectively, the full freedom for field steering and 3D spatial mapping of activity source.

The electrode array is an assembly of single electrode elements with the array having multiple non-planar channels. The electrode array is assembled in four stages of complexity:
1. a single electrode element containing one or more recording channels and one or more stimulating channels;
2. an element stack with one or more single electrode elements aligned along the element surfaces in a staggered fashion such that the contact pad of each element is exposed;
3. an electrode shank with an arrangement of one or more element stacks that face one or more directions; and
4. an electrode array which is an arrangement of electrode shanks such that the lateral separation of the shanks is perpendicular to the shanks.

An advantage of the invention is the ease of customizing the configuration of the electrodes. By keeping a small set of single elements, more complex electrode configurations can be assembled from this set of single elements. Depending on the requirements, the number of channels, the channel spacing, the complexity of assembly differs.

In one embodiment, referred to herein as a “Christmas tree” configuration, the electrode has two stacks facing opposite directions.

In another embodiment, referred to herein as an “Empire State Building” configuration, the electrode has four stacks facing different directions.

In yet another embodiment, the electrode has eight stacks facing different directions;

In yet another embodiment, the electrode has eight shanks by eight shanks, where each shank has 14 channels.

In yet another embodiment, the electrode has three shanks by three shanks, where each shank has 9 channels.

Further details and aspects of the invention will be described or will become evident in the course of the following detailed description.

### BRIEF DESCRIPTION OF THE DRAWINGS

Preferred and exemplary embodiments of the invention will now be described with reference to the accompanying drawings, in which:

FIG. 1 (prior art) shows the linear or planar channel arrangement of three existing neuromodulation electrodes;

FIG. 2 demonstrates the 4 stages of electrode assembly complexity;

FIG. 3 shows the different tuning parameters to get the desired channel arrangement;

FIG. 4 shows applications of non-planar electrodes;

FIG. 5 shows “Christmas tree” configurations;

FIG. 6 shows “Empire State Building” configurations;

FIG. 7 shows an eight-sided configuration;

FIG. 8 shows a eight-by-eight electrode array; and

FIG. 9 shows a three-by-three electrode array.

### DETAILED DESCRIPTION OF THE INVENTION

It should be understood that the following detailed description is of specific embodiments, as examples of the invention only. The invention is not restricted to the specific embodiments described and illustrated.

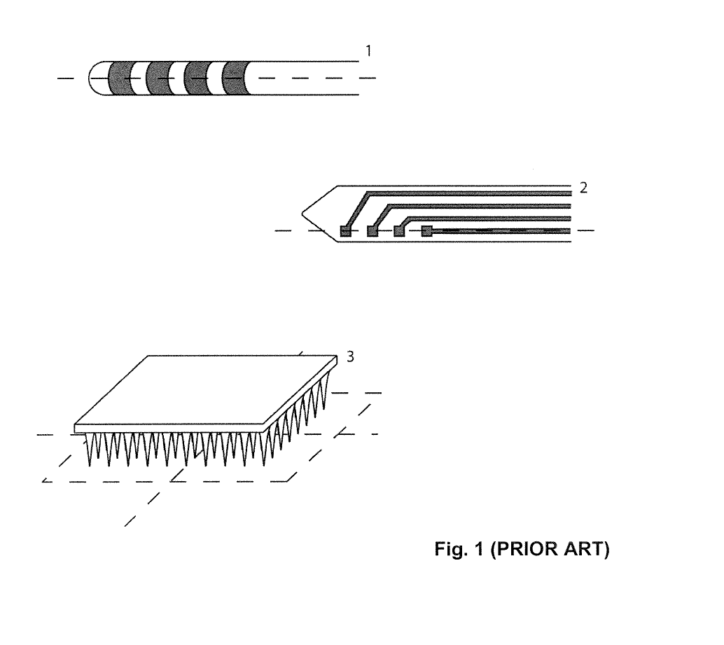

FIG. 1 (prior art) shows the linear or planar channel arrangement of existing neuromodulation electrodes.

The FIG. 1.1 electrode is made by Medtronic Inc. as the implantable distal end for deep brain stimulation. This electrode has four cylindrical channels linearly arranged along the central axis of the cylinders. The stimulation fields achieved with this electrode are limited to a sphere or an ovoid, whereas the target neuronal nuclei often have irregular shapes. The shape limitations of the stimulation field either causes inadequate coverage, which reduces the effectiveness of the therapy, or causes spillage into neighboring nuclei, which causes undesirable side-effects.

The FIG. 1.2 item is a multi-channel probe used often in animal neurophysiology research market. It is described in U.S. Pat. Nos. 6,330,466, and 6,829,498, and numerous publications. It has planar surface on which all the channels are arranged. For certain applications these planar channels are further positioned into a linear formation. When recording from a linear or planar channel arrangement, the source of activity can only be deduced as a projection on the line or the plane, respectively. A 3D spatial location can only be identified by a set of recording channels that can define the 3D volume.

The FIG. 1.3 item is a multi-channel electrode array used for brain computer interface (BCI), where the electrodes record at many sites to map the subject's neuronal activity and to correlate to the subject's intention. The signals are recorded in application specific regions of the cortex. For instance, to move a cursor, the signals are collected from the motor cortex. BCI needs the channels to be densely covering a 3D volume of neuronal tissue, or at the very least a 2D volume of neuronal tissue. This electrode is described in U.S. Pat. No. 5,214,088. Note that this electrode is an array of insulated pins whose exposed tips form a planar arrangement of channels. Thus, it cannot record from a volume of neurons.

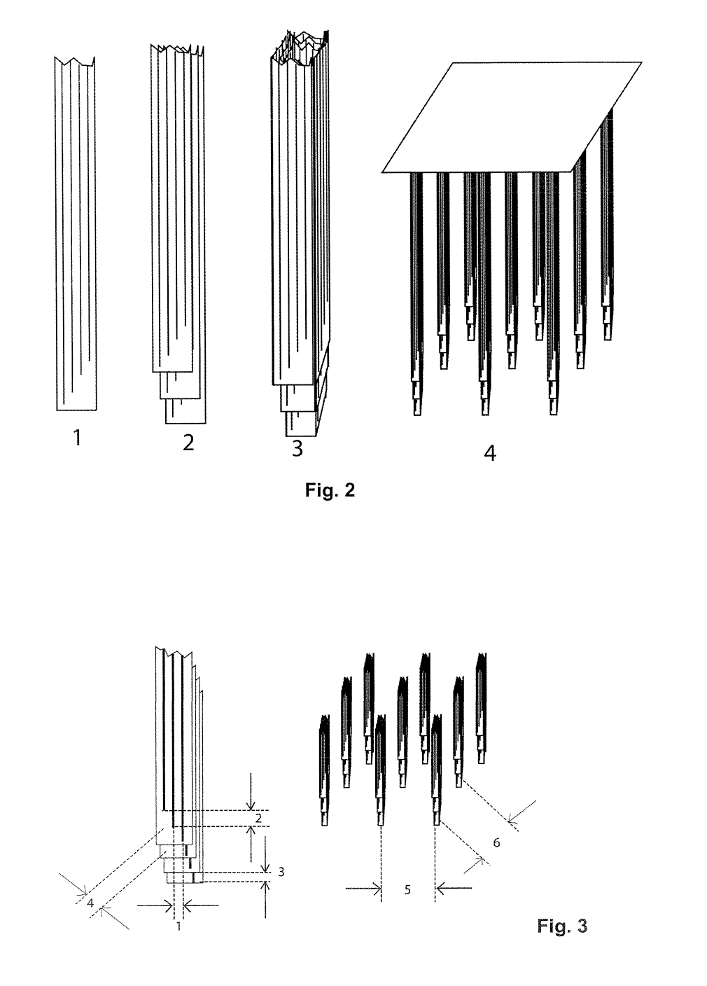

FIG. 2 demonstrates the four stages of electrode assembly complexity in the invention:
1. A single electrode element, which is the basic unit to be assembled. Each element contains one or more recording channels and one or more stimulating channels. For each channel, there is an insulated track body, an exposed contact pad, and possibly an exposed bonding pad or direct connection to an interconnect. One or more designs of the elements exist such that the set of the base units can construct the variety of electrodes configurations. In FIG. 2.1, each element has four channels.
2. An element stack which is one or more single electrode elements aligned along the element surfaces in a staggered fashion such that the contact pad of each element is exposed. In FIG. 2.2, there are three of the same elements stacked and glued together.
3. An electrode shank is an arrangement of one or more element stacks that face one or more directions. In FIG. 2.3, there are four stacks assembled with each facing one direction.
4. An electrode array is an arrangement of electrode shanks such that the lateral separation of the shanks is perpendicular to the shanks. In FIG. 2.4, there are nice shanks arranged in a 3-by-3 regular grid.

FIG. 3 shows the different tuning parameters to get the desired channel arrangement.

It is important to customize the distance between contact pads based on application, functional efficiency and manufacturing difficulties. There are several levels of distance-tuning parameters. Spatial arrangement of channels is useful in all three dimensions. Lateral distance between the channels on the same element and vertical distance due to staggering are two obvious dimensions. The third dimension in a grid formation is also clear. However, in an array, there is inherently some control in the third dimension. This control comes from the lateral distance caused by the thickness of succeeding elements.

Horizontal fine tuning 1 (FIG. 3.1) controls the inter-channel distance on a single element and it offers the best resolution (in one embodiment, the range is 10 to 50 microns). It is limited by the width of the element. Fine tuning requires the changes to be made during the pre-fabrication design stage.

Vertical fine tuning 2 (FIG. 3.1) has similar properties to the horizontal fine tuning. It is limited by vertical coarse tuning, because the channels need to be exposed. In one embodiment, it ranges between 5 to 100 microns

Vertical coarse tuning 3 (FIG. 3.1) controls the staggering distance between electrode elements in an electrode array. It has a relatively low resolution (in one embodiment 50 to 1000 microns), but the advantage is quicker customization by leaving this tuning to the assembly stage.

Depth tuning 4 (FIG. 3.1) can controls the lateral distance between neighboring electrode arrays. This parameter is limited by the thickness of the elements, and in one embodiment, the range may be 30 to 450 microns. This parameter is most difficult to adjust because it requires changes in the entire machining process. The lower limit arises from the insulation properties of the dielectric layers.

Horizontal coarse tuning 5 (FIG. 3.2) controls the distance between the shanks. This distance must be large enough not to impale or displace all the neuronal tissue that would be between the shanks. On the other hand, the distance must be small enough for channels to supplement each other in recording applications and interact with each other in stimulation applications.

Depth coarse tuning 6 (FIG. 3.2) is similar to horizontal coarse tuning with the same limitations. The two parameters are separated because they need not be the same in value.

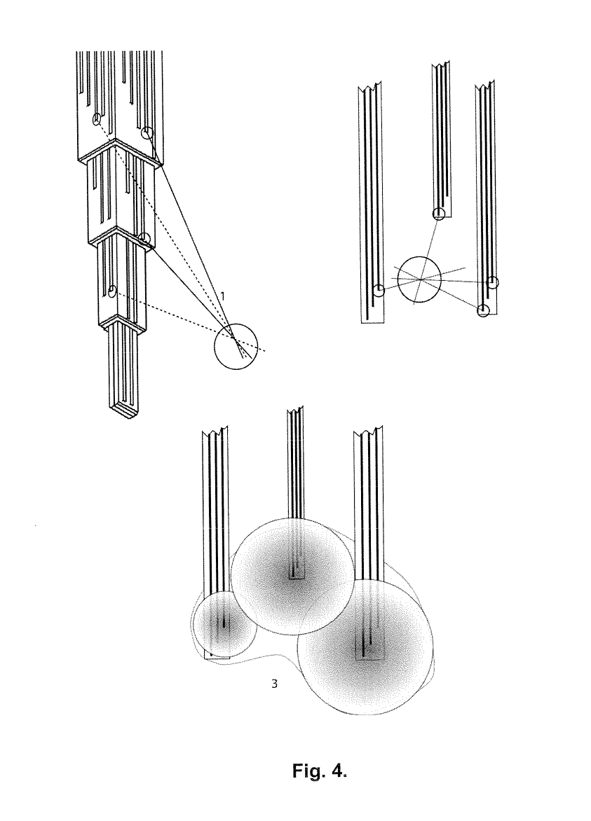

FIG. 4 shows applications of non-planar electrodes. Electrodes with non-planar channel arrangements have specific advantages.

Ref. 1 (FIG. 4.1) illustrates that when multiple channels are recording a particular neuron activity, spatial mapping algorithms can determine the exact location of the activity. In this case, the activity source is relatively close to one electrode shank, and the four selected recording channels are on one shank.

In FIG. 4.2, the activity source is too far away for the channels on one shank to localize accurately. Therefore, channels on various electrodes are selected such that the volume containing the activity source can be properly defined.

In FIG. 4.3, for stimulation, the target neuron cluster is of irregular shape. Multiple stimulating channels can receive different stimulation patterns such that the overall stimulation field conforms as much to the cluster as possible.

FIG. 5 shows a “Christmas tree” configuration. This embodiment has eight stimulation channels and five recording channels. Each element has a stimulating channels 1 and the recording channels 2. In this embodiment, the channels are made of platinum, specifically from platinum foil. The rest of the element consists of a dielectric layer that envelope the channels. The assembly of these layers uses thermo-bonding that activates an adhesive previously deposited on the dielectric layer.

The elements are stacked together such that the stagger exposes both channels. They are assembled using biocompatible glue cured by UV lamp. The elements are aligned in a fixture. Finally, the stacks are arranged back to back in two directions. A unique element in this stack is the recording only elements 5, which are used at the tip of the electrode such that the larger stimulating channel does not destroy the tissue to be recorded before the recording channel can reach them. The electrode is coated to reduce the rugged edges along the body of the electrode.

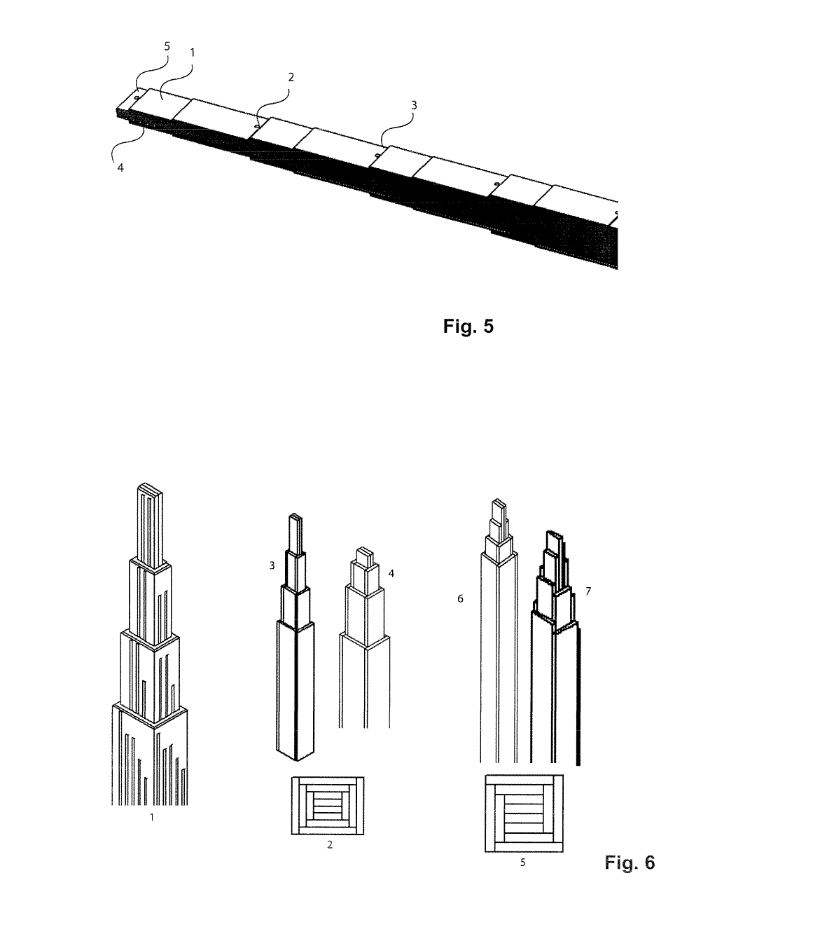

FIG. 6 shows the “Empire State Building” configuration. This embodiment has channels facing four directions (FIG. 6.1). One way of building a four-sided electrode is to arrange the elements in a rectangular configuration (FIG. 6.2). The advantage of this arrangement is that elements can be added to the shank in two directions at a time.

The elements can be staggered evenly (FIG. 6.3) which forms sets of channels at set heights. It facilitates the programmer's task because the heights of the channels are easily identified. Furthermore, channels at the same height can be activated simultaneously to mimic the action of a channel with a larger contact surface or a channel with oriented to multiple directions.

The elements can be staggered with a shift towards the tip (FIG. 6.4). This configuration is useful to bias the number of channels along the active region. Since the best option is to use the channels near the tip, thereby reducing unnecessary trauma, having more channels near the tip means that most of the configurability is offered in this region. This is a bias towards the tip. On the other hand, the shift can also balance the number of channels. Since the elements near the base of the electrode are much wider, they can accommodate more channels. By moving some of those channels up, the number of channels along the entire region would be even.

Another arrangement of the elements is in a square formation (FIG. 6.5). The advantage of this design is that the electrode is perfectly symmetrical, so that the orientation of the electrode is trivial during implantation. On the other hand, this electrode requires a change in orientation for each element, which is additional assembly time. Again it's possible to stagger the elements evenly (FIG. 6.6). It is also possible to stagger each element (FIG. 6.7), which is not possible with the rectangular arrangement. If each element can be staggered, then the increase in diameter is much more gradual and the same type of channel arrangement can be used on all the elements throughout.

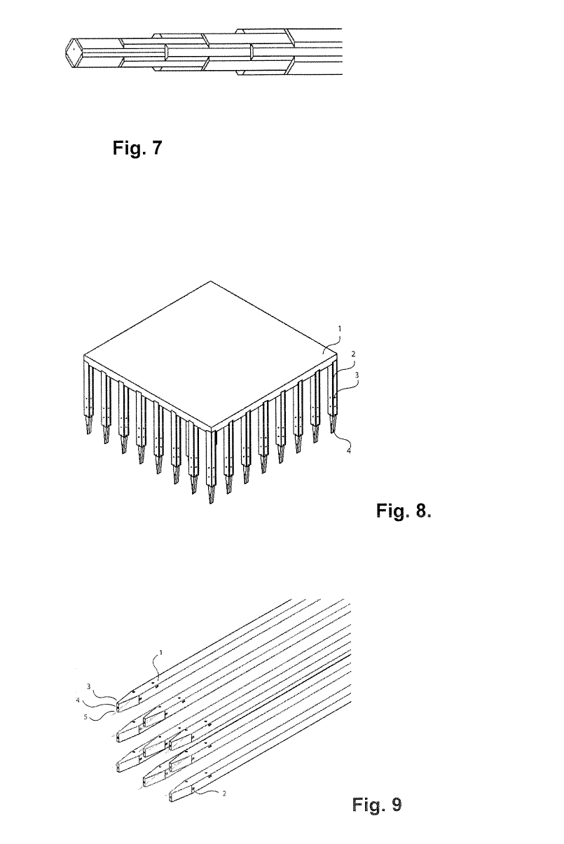

FIG. 7 shows a eight-sided configuration. This embodiment arranges elements in eight directions. The main advantage is that the electrode resembles more to a cylindrical or a conical shape. Fewer sharp corners mean less likelihood for trauma. In this design, a backbone is used to support the first elements. The elements have the surface corners cut off to form angles that are conducive to stacking in a 45-degree angle.

FIG. 8 shows an eight-by-eight electrode array. This embodiment has 64 shanks connected to a backbone 1. Each shank 2 has fourteen recording channels 3. The center of the shanks is composed of two elements with pointed 4 and these elements have one channel each. The outside of each shank consists of four elements each with three channels. The electrode is ideal to cover a volume of neurons in the cortex.

FIG. 9 shows a three-by-three electrode array. This embodiment has nine shanks connected to a backbone. Each shank 1 has nine recording channels 2. This electrode has three elements. The element on the top 3 has two channels along surface and one channel along the side. The element in the middle 4 has two channels along the side and one channel at the tip. The element on the bottom 5 has two channels along the bottom surface and one channel along the side. Therefore, each shank has two channels per side and one at the tip. This electrode has longer shank body to embed deeper into the cortex.

It should be appreciated that the above description relates to specific embodiments, and that many variations on the specific embodiments will be apparent to those knowledgeable in the field of the invention. The invention, as defined by the following claims, should not be restricted to any of the specific embodiments described above, which are merely examples of the invention.

1. A multi-channel electrode assembly for use in neuromodulation and the like, comprising multiple electrodes elements, the electrode elements arranged in a stack with their contact pads in a non-linear and non-planar fashion.

2. A multi-channel electrode assembly for use in neuromodulation and the like, comprising an array of multiple electrode shanks, each electrode shank comprising a multiple electrode stacks, each electrode stack comprising multiple electrode elements, the electrode elements arranged in the stack with their contact pads in a non-linear and non-planar fashion.

3. A multi-channel electrode assembly as in claim 2, arranged in a “Christmas tree” configuration.

4. A multi-channel electrode assembly as in claim 2, arranged in an “Empire State Building” configuration.

5. A multi-channel electrode assembly as in claim 2, arranged in an eight-sided configuration.

6. A multi-channel electrode assembly as in claim 2, arranged in an eight-by-eight electrode array.

7. A multi-channel electrode assembly as in claim 2, arranged in a three-by-three electrode array.
